(19) United States
(12) Patent Application Publication
(10) Pub. No.: US 2010/0074969 A1
(43) Pub. Date: Mar. 25, 2010

(54) METHOD OF CONTROLLING BLOOD SUGAR LEVELS, INSULIN LEVELS, CHOLESTEROL LEVELS, BODY FAT LEVELS, AND BODY WEIGHT BY ADMINISTERING A NUTRIENT FIBER MATRIX

(75) Inventors: Stewart F. Hughes, Orem, UT (US); Curtis A. Hedges, Highland, UT (US); G. Paulo Bangerter, American Fork, UT (US)

Correspondence Address: KIRTON AND MCCONKIE, 60 EAST SOUTH TEMPLE,, SUITE 1800, SALT LAKE CITY, UT, 84111, US

(73) Assignee: UNICITY INTERNATIONAL, INC., Orem, UT (US)

(21) Appl. No.: 12/563,556

(22) Filed: Sep. 21, 2009

Related U.S. Application Data
(60) Provisional application No. 61/098,540, filed on Sep. 19, 2008.

(51) Int. Cl. A61K 33/24; A61K 36/886; A61K 31/736; A61K 36/258; A61K 36/31; A61P 3/10; A61P 3/04; A61P 3/00

(52) U.S. Cl. 424/655; 424/744; 424/195.18; 424/728; 424/755

(57) ABSTRACT

A nutrient fiber matrix and associated methods that help control blood sugar levels is described. The matrix can control blood sugar in a variety of ways. In one possible example, the matrix blocks carbohydrates in a manner that reduces the body's ability to take the carbohydrates into its cells. In another example, the matrix acts to increase a person's energy level and feelings of satiety. Thus, the matrix can reduce the person's desire to consume more food and, thereby, help the person to keep from increasing his or her blood sugar levels by eating more. In still another example, the fiber matrix helps sugars to be absorbed over a longer period of time so as to smooth out and prevent spikes in blood sugar levels.

### CROSS-REFERENCE TO RELATED APPLICATIONS

This application claims the benefit of U.S. Provisional Application No. 61/098,540, filed Sep. 19, 2008, entitled “Method of Controlling Blood Sugar Levels, Insulin Levels, Cholesterol Levels, Body Fat Levels, and Body Weight by Administering a Nutrient Fiber Matrix,” the entire disclosure of which is hereby incorporated by reference.

### BACKGROUND OF THE INVENTION

The present invention relates in general to orally administrable compositions and associated methods that act to control blood sugar (e.g., glucose) levels in the subject (e.g., person) that ingests the compositions. More specifically, the present application discusses a nutrient fiber matrix that helps control blood sugar levels, and as a result, helps control insulin levels, cholesterol levels, body fat levels, and body weight in the subject that ingests the matrix.

Currently, there are several techniques for loosing body weight, including exercise routines and dieting. However, people often become frustrated with the amount of work and self-denial required by exercising and dieting as well as the sometimes slow and unimpressive weight-loss rate. In an attempt to lose weight, some people have turned to thermogenic stimulants. Generally, thermogenic stimulants are substances that act to raise a subject's metabolism of adipose tissue and generate heat. However, some such substances have shortcomings. For instance, some thermogenic stimulants have been believed to cause side effects that can range in gravity from mild discomfort to untimely death.

In another example, some people use meal replacements in an attempt to lose weight. However, such meal replacements may often have shortcomings as well. For example, some meal replacements may consist of low-calorie, filler foods that may not satiate their consumer. Accordingly, a subject who eats such meal replacements can continue to feel hungry or to have cravings, which can cause the subject to feel uncomfortable or to consume additional food.

Additionally, some dietary supplements, such as thermogenics and meal replacements, may not help a subject's body control blood sugar levels. Accordingly, a subject who consumes such supplements may suffer from uneven glucose levels, which can cause jitteriness, lack of energy, cravings, and hunger sensations. As the subject consumes additional food to decrease the hunger sensations, the subject's body can be caused to produce insulin, which can increase the body's uptake of sugar from the food and prevent the body from burning or metabolizing its own body fat.

It would be an improvement in the art to provide orally administrable compositions and methods that act to control blood sugar levels in the bloodstream. It would be a further improvement to provide orally administrable compositions and methods that act to control a subject's insulin levels, cholesterol levels, body fat levels, and body weight, without adversely affecting the subject's health. Additionally, it would be an improvement to provide orally administrable compositions and methods that help a subject feel energetic and satiated. Such methods and compositions are provided herein.

### BRIEF SUMMARY OF THE INVENTION

The present invention relates to an orally administrable nutrient fiber matrix that acts to control blood sugar levels in a subject who ingests it. As part of, or in addition to the matrix's ability to control blood sugar levels, the matrix may also act to control insulin levels, cholesterol levels, body fat levels, and/or body weight in the subject that ingests the matrix.

The nutrient fiber matrix may contain any ingredient that allows the matrix to help regulate blood sugar levels when ingested by humans. Generally, however, the fiber matrix comprises a blend of fibers, plant extracts, vitamins, minerals, and/or other biologically acceptable compounds, such as flavorings, phytonutrients, excipients, carriers, etc. Some non-limiting examples of suitable fibers include guar gum, gum Arabic, locust bean gum, citrus pectin, oat fiber, beta glucan, and other pharmacologically-acceptable fibers. Some non-limiting examples of suitable plant extracts include phaseolamine (common bean extract), broccoli powder, Aloe vera, hydroxypropyl methylcellulose, ginseng, and other biologically-acceptable plant-derived substances. Some non-limiting examples of vitamins and minerals may include calcium carbonate, maltodextrin, vitamin C, vitamin A, vitamin E, Niacin, zinc, folic acid, biotin, vitamin B1, vitamin B2, vitamin B6, vitamin B12, sodium selenite, chromium (e.g., chromium (III) chloride hexahydrate), and the like.

The nutrient fiber matrix can be administered in virtually any biologically-acceptable and orally administrable form, including as powder, a pill, a tablet, a gel-cap, an elixir, a syrup, a food product, a drink, or the like. In some instances, however, the powder form is preferred. In such instances, the matrix can be ingested in any suitable manner. For example, the matrix may be mixed with a liquid (e.g., water, juice, milk, or beverage) and be taken as a drink.

In some instances, a subject ingests the matrix before eating a meal. In such instances, the matrix can help a subject feel full faster. The matrix may also act to sequester carbohydrates and nutrients or otherwise slow the rate at which carbohydrates and nutrients are absorbed into the body. Additionally, the fiber matrix may help to push carbohydrates through the digestive tract faster and, thereby, reduce the amount of carbohydrates that are absorbed by the body.

In an example of a typical method for using the fiber matrix in a 24-hour period, the fiber matrix is ingested within about 30 minutes (e.g., about 5-15 minutes) before at least 1 meal and preferably before 2 or 3 meals. Moreover, the fiber matrix may be most effective at controlling blood sugar levels and levels of insulin, cholesterol, body fat, and body weight if the matrix is taken before or in conjunction with a subject's highest carbohydrate meal(s) of the day. Additionally, the fiber matrix has been found to be more effective when the subject consuming the matrix does one or more of the following: eats a high-protein, low-carbohydrate breakfast; eats small well-balanced meals; reduces the amount of processed foods and simple carbohydrates consumed by the subject; does little to no snacking between meals; and spaces meals apart by approximately 4 hours or more. As a non-limiting example of a method for spacing meals, the subject may wait about 4 hours after the first meal of the day (e.g., breakfast) before eating another meal (e.g., lunch). Similarly, the subject may wait about 4 hours or more after lunch before eating the final meal of the day (e.g., dinner). After the last meal of the day, the subject may wait about 12 hours before consuming another meal (e.g., breakfast).

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussions of the features and advantages, and similar language, throughout this specification may, but do not necessarily, refer to the same embodiment, but may refer to every embodiment.

Furthermore, the described features, advantages, and characteristics of the invention may be combined in any suitable manner in one or more embodiments. One skilled in the relevant art will recognize that the invention may be practiced without one or more of the specific features or advantages of a particular embodiment. In other instances, additional features and advantages may be recognized in certain embodiments that may not be present in all embodiments of the invention. Additionally, while the invention has been found to be effective on humans, the skilled artisan will also recognize that the nutrient fiber matrix and its associated methods may also prove beneficial for use on a variety of other mammals.

These features and advantages of the present invention will become more fully apparent from the following description and appended claims, or may be learned by the practice of the invention as set forth hereinafter.

### BRIEF DESCRIPTION OF THE DRAWINGS

FIG. 1 contains a table illustrating suitable ranges for ingredients of some embodiments of a nutrient fiber matrix; and

FIG. 2 contains a flow chart illustrating a representative embodiment of a method for ingesting the nutrient fiber matrix and spacing meals.

### DETAILED DESCRIPTION OF THE INVENTION

It is generally understood that as a subject (e.g., a person) consumes food, the subject's blood sugar levels increase. To some extent, the increased blood sugar levels cause the subject's pancreas to produce insulin. This insulin works to move sugars from the subject's blood into the subject's cells. Additionally, insulin prevents the subject's body from burning its own fat stores for energy, which can cause the subject to feel hungry more often.

The present application discusses a nutrient fiber matrix and associated methods that help control blood sugar levels in a subject who consumes the matrix. The nutrient fiber matrix can control blood sugar in a variety of ways. In one possible example, the matrix acts to increase a feeling of satiety and energy in the subject who consumes it. Thus, the matrix can reduce the subject's desire to consume more food and, thereby, help the subject to keep from increasing his or her blood sugar levels by eating more. As a result, the subject's body produces less insulin and metabolizes more of the body's fat stores. As body fat is burned, the subject feels additional energy and less hunger feelings. Accordingly, the subject can comfortably go for longer periods of time without eating than would otherwise be possible. In another example, the matrix blocks carbohydrates in a manner that reduces the body's ability to take the carbohydrates into its cells. In still another example, the fiber matrix helps sugars and nutrients to be absorbed over a longer period of time so as to smooth out and prevent spikes in blood sugar or nutrient levels. Under a non-binding theory, as the nutrient fiber matrix regulates the body's blood sugar levels, additional metabolic levels, such as body fat levels, cholesterol levels, body weight levels, and/or levels of substances that support the subject's general health and well being may be controlled.

Generally, the nutrient fiber matrix may comprise any biologically-acceptable ingredient that allows the matrix to control blood sugar levels in a subject who consumes it. As used herein, the term “biologically-acceptable” refers to a substance that is safe for human consumption. Additionally, the term “control” can be used herein to express the idea of reducing, balancing, managing, stabilizing, modulating, and/or otherwise regulating a biological characteristic (e.g., a blood sugar level) in a beneficial manner. Some examples of suitable ingredients in the nutrient fiber matrix include, but are not limited to, a variety of fibers, plant extracts, vitamins, minerals, and/or other biologically-acceptable ingredients that allow the matrix to control a subject's glucose levels.

The nutrient fiber matrix may comprise a variety of biologically-acceptable soluble and/or non-soluble fibers that allow the matrix to control a subject's blood sugar levels. In some embodiments, the matrix comprises guar gum, gum Arabic, locust bean gum, citrus pectin, oat fiber, maltodextrin, and/or beta glucan. Indeed, while the matrix can comprise any of these fibers, in any suitable combination, in some embodiments, the matrix comprises all of these fibers. In addition to the aforementioned fibers, the matrix optionally comprises any other suitable fiber, including, but not limited to, psyllium, gum acacia, xanthane gum, innuline, fructo-oligosaccharides (FOS), carrageenan, and combinations thereof. In some preferred embodiments, the matrix comprises both soluble fibers (including, but not limited to, guar gum, gum Arabic, locust bean gum, pectin, oat fiber, beta glucan, psyllium, gum acacia, xanthane gum, innuline, fructo-oligosaccharides, carrageenan, and/or other carbohydrates that are resistant to digestion and absorption in the human small intestine and that are capable of completely or partially being fermented in the large intestine) and insoluble fibers.

The various fiber ingredients of the matrix may provide or improve several metabolic functions in the subject who consumes the matrix. By way of non-limiting example, the various fibers can act to control blood glucose levels, control insulin levels, reduce carbohydrate absorption, lower total serum cholesterol, lower serum LDL levels, increase feelings of fullness, decrease feelings of hunger, maintain regular bowel movements, and soften stool. Additionally, the fiber ingredients may also help to reduce blood pressure, reduce arteriosclerotic plaque, increase HDL cholesterol, help cardiac output, and/or increase the pumping strength of the heart. In one example of how the various fibers may work, it is believed that the fiber matrix acts to smooth the absorption of carbohydrates, so that the subject's blood sugar level remains more constant.

As previously mentioned, the fiber matrix also comprises a variety of plant extracts. Indeed, in some embodiments, the matrix comprises one or more of the following: phaseolamine (common bean extract), broccoli powder, Aloe vera, hydroxypropyl methylcellulose (HPMC), and ginseng. Indeed, in some preferred embodiments, the matrix includes each of the aforementioned plant extracts. Of course, in addition to the aforementioned plant extracts, the nutrient fiber matrix may comprise any other biologically-acceptable, plant-derived substance that allows the matrix to control a subject's glucose levels.

Each of the aforementioned plant extracts may provide the matrix with one or more beneficial characteristics. In one example, it is theorized that the common bean extract acts to block carbohydrates, or at least reduce the rate at which carbohydrates are metabolized and absorbed by the body. Accordingly, the common bean extract may lower the amount of carbohydrates that are metabolized and taken into the subject's cells as well as help to maintain a more constant blood sugar level in the subject. In another example, it is believed that the common bean extract acts to stimulate the body in a manner that reduces the subject's food cravings. In yet another example, it is believed that ginseng causes the subject to have improved blood flow. In still another example, it is believed that Aloe vera acts to stimulate the bowels to reduce or prevent cramping. In still another example, the HPMC causes the nutrient fiber matrix to have a high viscosity when the matrix is mixed with a liquid. For instance, the HPMC can help the matrix to have a gel-like form when the matrix is introduced to the subject's stomach. Accordingly, the matrix may cause the subject to feel satiated.

In some embodiments, the nutrient fiber matrix also comprises a variety of vitamins and/or minerals. Some non-limiting examples of suitable vitamins and/or minerals may include, but are not limited to, vitamin B1 (thiamine HCl), vitamin B2 (riboflavin), vitamin B6 (pyridoxine HCl), vitamin B12 (cyanocobalamin), biotin, and chromium (e.g., chromium (III) chloride hexahydrate, chromium chloride, chromium sulfate, chromium enriched yeast, chromium polinicotinate, chromium piolinate, and mixtures thereof). While the matrix can comprise any suitable combination of the aforementioned vitamins and minerals, in some embodiments, the matrix preferably comprises all of the aforementioned vitamins and minerals. Moreover, the matrix may also comprise any other suitable vitamin or mineral that allows the matrix to control glucose levels. For example, the matrix may also comprise calcium (e.g., calcium carbonate), maltodextrin, vitamin B3, vitamin C (ascorbic acid), vitamin A (beta carotene), vitamin E (d-alpha-tocopheryl acetate), niacin (niacinamide), zinc (zinc gluconate), folic acid, sodium selenite (selenium), and/or other biologically-acceptable vitamins or minerals.

The various vitamins and minerals may also provide the fiber matrix with several beneficial characteristics. For example, the vitamins and minerals may help control blood glucose levels, reduce fat, raise HDL-cholesterol levels, reduce homo-cysteine levels, fight free radicals, and provide the subject with increased energy. For instance, it is believed that chromium may increase the subject's fat metabolism and, therefore, reduce fat levels in the subject.

The combination of vitamins, minerals, plant extracts, and fiber in the nutrient fiber matrix may work together to synergistically provide the matrix with benefits that may not be achieved by ingesting each at a non-overlapping time period. For example, where vitamins and/or minerals are administered with the fiber in the fiber matrix, the vitamins and minerals may counteract vitamin and mineral chelation that can be caused by insoluble fiber in the matrix. In another example, the fiber and/or plant extracts can act to release the vitamins, minerals, and/or other nutrients, such as those found in the plant extracts, into the body with a controlled release. As used herein, the term “controlled release” refers to the gradual release of the nutrient over a prolonged period of time. The term controlled release may include other terms, such as timed release, prolonged release, sustained release, or delayed release. Controlled release can further be described as producing a blood concentration curve of the nutrient substance that is broader than the curve produced without fiber and/or plant extract (e.g., HPMC), but with substantially equal area under the curve.

In addition to the previously mentioned ingredients, the nutrient fiber matrix may also comprise any other ingredient that allows the matrix to help control blood sugar levels, insulin levels, cholesterol levels, body fat levels, and/or body weight in the subject that ingests the matrix. By way of example, the matrix may comprise one or more flavorings; phytonutrients; antioxidants; substances that support the subject's immune system, cardiovascular system, etc.; coloring agents, scents; and/or other suitable ingredient.

Where the matrix includes one or more flavorings, the flavorings can comprise any suitable ingredient that provides the matrix with a desired taste. For instance, the matrix can comprise an orange juice flavoring, citric acid, sucralose, and/or another suitable ingredient that flavors the matrix.

As previously stated, in some embodiments, the matrix comprises phytonutrients. As used herein, the term “phytonutrients” may include plant derived compounds that have biological activity in the body. Phytonutrients naturally occur in vegetables and fruit. In broad terms, they can be said to be any biologically useful chemical or nutrient derived from a plant source. However, in common usage, they have a more limited definition. They are usually used to refer to compounds found in plants that are not required for normal functioning of the body but that nonetheless have a beneficial effect on their consumer's health or an active role in the amelioration of disease in their consumer.

Some phytonutrients found in fruits and vegetables have previously been classified as vitamins. For example, flavonoids were known as vitamin P, cabbage factors (glucosinolates and indoles) were called vitamin U, and ubiquinone was vitamin Q. Tocopherol remained on the list as vitamin E. The “vitamin” designation was dropped for the other nutrients because specific deficiency symptoms could not be established. Today scientists and researchers may group phytonutrients into classes on the basis of similar protective functions as well as individual physical and chemical characteristics of the molecules. Some examples of different classes of phytonutrients include, but are not limited to, terpenes, carotenoids, limonoids, phytosterols, phenols, flavonoids, anthocyanidins, catechins, gallic acids, isoflavones, thiols, glucosinolates, allylic sulfides, indoles, isoprenoids, tocotrienols, tocopherols, lipoic acids, ubiquinones, and phytoestrogens.

Some non-limiting examples of fruits and vegetables that contain phytonutrients may include, but are not limited to, acai, alfalfa, apple, artichoke, apricot, asparagus, avocado, barley grass, bilberry, beans, bittermelon, beet, blackberry, broccoli, black current, Brussels sprout, blueberry, cabbage, cantaloupe, cassaya, carrot, cherry, cauliflower, coconut, celery, coriander, cranberry, chlorella, guavas, corn, grape, cucumber, garlic, grapefruit, horseradish, hops, kale, kava, kamut, kiwi, lima beans, lemon, oat grass, mangos, olive, orange, parsley, papaya, peach, peas, pear, pepper, pineapple, potato, plum, pumpkin, pomegranate, rice, raspberries, spinach, strawberry, spirulina, tangerines, squash, tomato, sweet potatoes, wheat germ, wheat grass, white kidney beans, and mixtures thereof.

In some embodiments, the fiber matrix also comprises antioxidants, which may include vitamins and phytonutrients. Such antioxidants may be selected from, but are not limited to, lycopene, anthocyanosides, alfalfa chlorophyll complex beta-carotene, alpha-carotene, lutein, zeaxanthin, canthaxanthin, astaxanthin, tocopherol, epigallocatechin gallate (EGCG), acetylcysteine, alpha lipoic acid, beta carotene, bilberry, burdock, carnosine, catalase, conjugated linoleic acid (CLA), CoEnzyme Q10, cryptoxanthin, curcumin, daidzein, dehydroepiandrosterone (DHEA), dimethylaminoethanol (DMAE), garlic, genistein, germanium, Ginkgo biloba, glutamine, glutathione, grape seed extract, green tea, lutein, lycopene, manganese, melatonin, methionine, para-aminobenzoic acid (PABA), pycnogenol, quercetin, resveratrol, selenium, superoxide dismutase, taurine, vitamin C (ascorbic acid), vitamin E, zeaxanthin, zinc, and mixtures thereof.

In some embodiments, the fiber matrix comprises phytonutrients that include antioxidant plant enzymes. Such antioxidant plant enzymes may be selected from, but are not limited to, catalase, glucose oxidase, peroxidase, superoxide dismutase, glutathione peroxidase, and mixtures thereof. In at least one embodiment, the matrix comprises nutritional vitamins and phytonutrients that include antioxidant vegetable concentrates.

In some embodiments, the fiber matrix includes substances that support the subject's immune system. Substances that support the immune system may be selected from, but are not limited to, alfalfa leaf, alpha lipoic acid, allium cepa, Aloe vera, antioxidant plant enzymes, apricot extract, nectarine extract, arabinogalactan, Arnica montana, arsenicum album, bee pollen, benzenum, belladonna, beta carotene, biotin, Piper nigrum L (black pepper) extract, Piper longum L (long pepper) extract, bladderwrack (kelp) extract, boron, colostrum, burdock root, cadmium sulphuricum, calcium ascorbate, calcium citrate, capsicum, carotenoids, carrot, cat's claw, cayenne, chlorum, choline bitartrate, polynicotinate, citrus bioflavonoids (lemon), cruciferous vegetable concentrate, cupric oxide (copper), cuprum metallicum, copper glycinate, dandelion root, Drosera rotundifolia, echinacea, elderberry, ferrum phosphoricum, folic acid (folate), fructose, garlic, ginger root, golden seal root, grape seed extract, grape wine concentrate, green tea extract (leaf), guarana extract, xanthan gum, hops strobile, inositol, iron rice chelate, lactoferrin, lutein, lycopene, magnesium, manganese rice chelate, marigold flower extract, milk thistle herb, mixed berry anthocyanosides, molybdenum, Morinda citrifolia extract, mulberry, n-acetyl cysteine, nettle leaf, niacin/niacinamide, nux vomica, oxo vanadium bis glycinato, pantothenic acid, para amino benzoic acid, perilla seed extract, pine bark extract, plumbum metallicum, pomegranate extract, potassium, potassium iodide (iodine), prune extract (fruit), quercetin, red clover blossom, red wine extract, rhubarb root, rose hips, rutin, Schisandra chinensis fruit extract, scullcap herb, selenium, sheep sorrel herb, slippery elm bark, soy, tabacum, turmeric, valerian root, vitamin A palmitate, vitamin B1/B2, vitamin B6/B12, vitamin C (ascorbic acid), vitamin D (2&3), vitamin E (d-alpha-tocopherol), vitamin K (phytonadione), watercress leaf, CoEnzyme Q10, glutamine, hydroxymethylbutyrate (HMB), L-arginine, lentinan, red yeast, S-adenosyl-L-methionine (SAMe), sangre de grado (dragon's blood), whey protein, medium chain triglycerides (MCT), St. John's Wort, boxwood, dehydroepiandrosterone (DHEA), riboflavin (vitamin B2), Uña de Gato extract, zinc, and mixtures thereof.

In other embodiments, the nutrient fiber matrix includes substances that support the subject's cardiovascular system. Substances that support the cardiovascular system may be selected from, but are not limited to, chrysanthemum morifolium, acerola, apricot extract, nectarine extract, B-vitamins (e.g., 1, 2, 3, 6, and/or 12), beta carotene, biotin, black current seed oil, gamma-linolenic acid (GLA), borage oil, calcium phosphate, carnosine, carnithine, carotenoids, choline bitartrate, chondroitin sulfate, coconut oil, cognis phytosterols, copper (cupric oxide), CoEnzyme Q10, D-ribose, evening primrose seed oil, fish oil, eicosapentaenoic acid (EPA), docosahexaenoic acid (DHA), flax seed oil, folic acid (folate), garlic, grape seed extract, grape juice extract, grape skin extract, green tea extract, glutathione (GSH), hawthorne berry extract, hesperidin, inositol, isoleucyl-prolyl-proline (IPP), L-arginine, L-carnitine, L-cysteine, L-lysine, L-proline, L-taurine, lecithin, magnesium, manganese, molybdenum, niacin and niacinamide (vitamin B3), omega-3 fatty acids, pantothenic acid, pine bark extract, policosanols, pomegranate extract, potassium, protein, prune extract (fruit), quercetin, red wine extract, red wine (resveratol), rose hips, rutin, selenium, soy, turmeric, valyl-prolyl-proline (VPP), vitamin C (ascorbic acid), vitamin D, vitamin E (d-alpha-tocopherol), zinc, and mixtures thereof.

In some embodiments, the biologically active substance includes substances that support the subject's cardiovascular system by helping to reduce cholesterol. Substances that help to reduce cholesterol may be selected from, but are not limited to, niacin and niacinamide (vitamin B3), beta-sitosterol, flaxseed, red yeast, sitostanol, alfalfa, artichoke, avocado, barley, calcium, English walnut, green tea, jiaogulan, macadamia nut, magnesium, olive, rice bran, safflower, sitostanol, soy, soybean oil, sweet orange, yoghurt, amaranth, cod liver oil, garlic, guggul, inulin, lecithin, red clover, kefir, activated charcoal, aloe, bean pod, chitosan, cocoa, docosahexaenoic acid (DHA), fenugreek, flaxseed oil, glucomannan, hydroxymethylbutyrate (HMB), hyperimmune egg, inositol nicotinate, Job's Tears (Coix lacryma-jobi), policosanol, pomegranate, pycnogenol, quercetin, royal jelly, sunflower oil, vitamin C (ascorbic acid), vitamin E, vitamin K, yucca, and mixtures thereof.

In some embodiments, the nutrient fiber matrix includes substances that support the subject's cardiovascular system by helping to reduce blood pressure. Substances that help to reduce blood pressure may be selected from, but are not limited to, alpha-linolenic acid, calcium, cod liver oil, CoEnzyme Q10, fish oil, garlic, green tea, olive, Oolong tea, potassium, pycnogenol, stevia, sweet orange, vitamin C (ascorbic acid), wheat bran, eicosapentaenoic acid (EPA), gamma linolenic acid (GLA), vitamin E, casein peptides, cocoa, dimethylsulfoxide (DMSO), grape, hydroxymethylbutyrate (HMB), L-arginine, soy, tomato, yucca, guggul, alpha-linolenic acid, kefir, activated charcoal, aloe, bean pod, chitosan, cocoa, docosahexaenoic acid (DHA), fenugreek, flaxseed oil, glucomannan, hydroxymethylbutyrate (HMB), hyperimmune egg, inositol nicotinate, Job's Tears (Coix lacryma-jobi), policosanol, pomegranate, pycnogenol, quercetin, royal jelly, sunflower oil, vitamin C (ascorbic acid), vitamin E, vitamin K, yucca, and mixtures thereof.

In some embodiments, the nutrient fiber matrix includes substances that support the subject's cardiovascular system by helping to reduce arteriosclerotic plaque. Substances that help to reduce arteriosclerotic plaque may be selected from, but are not limited to, alpha-linolenic acid, black tea, fish oil, garlic, niacin and niacinamide (vitamin B3), vitamin C (ascorbic acid), vitamin E, Gotu kola, lycopene, mesoglycan, pomegranate, stevia, sweet orange, wheat bran, and mixtures thereof.

In some embodiments, the nutrient fiber matrix includes substances that support the subject's cardiovascular system by helping to lower LDL cholesterol and/or increase HDL cholesterol. Substances that help to lower LDL cholesterol and/or increase HDL cholesterol may be selected from, but are not limited to, niacin, beta-sitosterol, flaxseed, red yeast, alfalfa, artichoke, avocado, hyperimmune egg, barley, calcium, English walnut, garlic, green tea, jiaogulan, macadamia nut, magnesium, olive, inositol nicotinate, policosanol, rice bran, safflower, sitostanol, soy, soybean oil, sweet orange, yoghurt, amaranth, cod liver oil, guggul, inulin, lecithin, red clover, kefir, activated charcoal, aloe, bean pod, chitosan, docosahexaenoic acid (DHA), fenugreek, glucomannan, hydroxymethylbutyrate, pomegranate, pycnogenol, quercetin, royal jelly, sunflower oil, vitamin C, vitamin E, vitamin K, yucca, and mixtures thereof.

In some embodiments, the nutrient fiber matrix includes substances that support the subject's cardiovascular system by helping to improve cardiac output and/or increase the pumping strength of the heart. Substances that help to improve cardiac output and/or increase the pumping strength of the heart may be selected from, but are not limited to, hawthorne berry, CoEnzyme Q10, taurine, carnithine, and mixtures thereof.

In still other embodiments, the nutrient fiber matrix includes substances that further help to control the subject's body weight. Substances that may help to control the subject's body weight may be selected from, but are not limited to, almond, Aloe vera, alpha lipoic acid, aminogen, ammonium glycyrrhizate, amylum fruit extract, astaxanthin, bean pod, benzyl alcohol, biotin, bitter orange, Piper nigrum L (black pepper) extract, Piper longum L (long pepper) extract, black tea extract, bladderwrack (kelp), blue-green algae, broccoli, butylene glycol, Indian Fig Opuntia cactus, caffeine, caralluma, carob, cassia seed extract, cayenne, calcium, calcium phosphate, cedarwood oil, cetyl alcohol, chitosan, HD, Cissus quadrangularis extract (stem & leaves), citrus lime oil, citrus orange oil, cocoa, CoEnzyme Q10, coix seed, cola nut, Coleus forsholii extract, cujquat (fruit), Combreturn micranthum (leaf) extract, copper sulfate, cyclometicone, dandelion root, dehydroepiandrosterone (DHEA), 7-keto-DHEA, dill weed, diacylglycerol, dimethicone, disodium succinate, DL-phenylalanine, ephedra, flos citri auranti (blossoms), folic acid, Garcinia cambogia extract, geranium oil, ginger root, American root, fish oil, ginseng, panax extract, glyceryl stearate, grapefruit oil, green tea extract (leaf), guaiacwood oil, guarana extract, Gymnema sylvestre, Hoodia gordonia (stem), glucomannan, green tea, guggul, 5-hydroxytryptophan (5-HTP), inulin, kahkow fruit extract, lavendin oil, lecithin, hydroxylated, lemon grass, licorice, linoleic acid, L-carnitine, L-glutamine, L-methionine, L-tyrosine, Lespedeza capitata extract, Litsea cubeba fruit oil, lotus leaf, magnolia, manganese, methyl paraben, milk protein isolate, mulberry (leaf), nettle leaf, niacin/niacinamide, Oolong tea extract (Camellia sinensis), pantothenic acid, papaya leaf, PEG-12/PEG-100, phaseolamine, phellodendron, picamilon HCl, pine leaf oil, potassium citrate, potassium iodide (iodine), potassium phosphate, Poria cocos (Fu Ling), propylparaben, pyruvate, quercetin, red clover blossom, Rhodiola rosea extract, rhubarb root (Da Huang), Rooibos tea extract (leaf & stem), rosemary leaf oil, sesame oil (Sesamum indicum), senna (leaf), Caralluma fimbriata, sodium benzoate, sodium caseinate, soy lecithin, soy protein isolate, Spanish sage oil, stevia leaf, sunflower oil, tangerine oil, tarragon extract, theobromine, threonine triethanolamine, tiratricol, Ulva lactuca extract, Arctostaphylos uva ursi leaf, vinpocetine, vitamin A, vitamin B1/B2, vitamin B6/B12, vitamin C (ascorbic acid), vitamin D3, vitamin E (d-alpha-tocopherol), water plantain rhizome (stem), whey powder, whey protein isolate, white willow bark extract, white pepper, Withania somnifera extract, Wu-Long tea, Yellowdock root, Yerba mate extract, zinc oxide, and mixtures thereof.

In still other embodiments, the nutrient fiber matrix can comprise any suitable therapeutic substance; metabolite; medicament; substance used for the treatment, prevention, diagnosis, cure, or mitigation of disease or illness; substance that affects anatomical structure or physiological function; and/or substance that alters the impact of external influences on an animal. For a more detailed description of possible ingredients that can be added to a fiber matrix, see U.S. patent application Ser. No. 11/759,410, entitled “Method and Composition for Providing Controlled Delivery of Biologically Active Substances,” filed Jun. 7, 2007; the entire disclosure of which is hereby incorporated by reference.

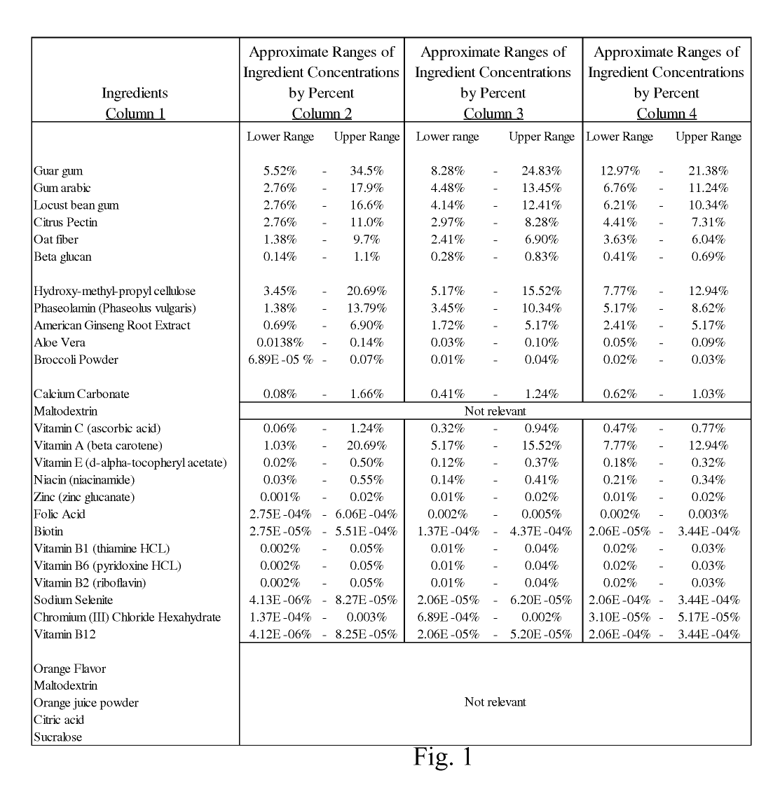

Each of the ingredients in the nutrient fiber matrix can be present in a dosage unit at any percent that is biologically-acceptable and allows the matrix to help control the subject's glucose levels. Nevertheless, it has been found that the various ingredients can act synergistically and perform optimally when the matrix comprises specific percents of the various ingredients. For example, FIG. 1 contains a table depicting some non-limiting examples of suitable ingredient concentrations, by percent, in typical dosage units of the fiber matrix. In FIG. 1, the first column lists suitable ingredients. According to some embodiments, the second column lists a corresponding concentration range (in percent) for each of the ingredients in the first column. Similarly, the third and fourth columns each list suitable concentration ranges, according to various embodiments of the fiber matrix. In sum, each ingredient listed in the first column of FIG. 1 may be present in the fiber matrix at a concentration selected from the second third or fourth column, as long as the combination of all the ingredient concentrations together, do not exceed 100%. It will be understood that if some of the ingredients listed in Table 1 are not present, that the relative concentration amounts of the other ingredients can be adjusted accordingly.

The nutrient fiber matrix may be administered to the subject in any suitable size dosage unit. In some embodiments, a dosage unit weighs between about 1 and about 56 grams. In other embodiments, a dosage unit weighs between about 3 and about 20 grams. In still other embodiments, a dosage unit weighs between about 6 and about 15 grams (e.g., about 7.25 grams±1 gram).

The subject may take any biologically-acceptable amount of the nutrient fiber matrix during a desired time period. In fact, in some embodiments where the subject is an average size adult, it may be beneficial for the subject to ingest between about 3 and about 56 grams (e.g., between about 10 and about 45 grams) of the matrix each day. In other embodiments, an adult subject may choose to consume between about 6 and about 40 grams of the matrix in a day. In still other embodiments, an adult subject may consume between about 14 and about 30 grams of matrix in a day. Of course, the skilled artisan would recognize that the amount of nutrient fiber matrix consumed each day may be modified for the specific characteristics of the subject (e.g., weight, size, age, health, sex, etc.).

The nutrient fiber matrix can be manufactured in practically any biologically-acceptable and orally administrable form, including as powder, a pill, a tablet, a gel-cap, an elixir, a syrup, a food product, a drink, or the like. In some instances, however, the powder form is preferred. In such instances, the matrix can be ingested in any suitable manner. For example, the matrix may be mixed with a liquid (e.g., water, juice, milk, etc.) and be taken as a drink. In this example, the fiber matrix can be ready to interact with food in the subject's digestive tract as soon as the matrix is ingested. Additionally, when taken as a drink, the matrix may be easily ingested.

The nutrient fiber matrix may be used in any manner that allows the matrix to control glucose levels in the subject. In some embodiments, however, it may be beneficial to consume the matrix substantially simultaneously with or prior to eating a meal. As used herein the term “substantially simultaneous with a meal” may mean during, at the beginning of, or at the end of the subject's meal. Also, as used herein, the term “meal” may refer to any of the occasions for eating food that occur by custom or habit, at more or less fixed times, including, but not limited to, breakfast, lunch, and dinner. The term “prior to eating” is intended to exclude and distinguish administering the oral dosage unit to the subject after a meal or while the subject is eating the meal. The term “prior to eating” will typically mean within about 30 minutes before eating the meal (e.g., between about 5 and about 15 minutes before eating).

When the nutrient fiber matrix is taken with or before a meal, the matrix may provide several benefits. In one example, when taken before a meal, the matrix can help the subject feel full faster than the subject would have felt without ingesting the matrix. Accordingly, the subject may be more likely to consume less. In another example, when taken before a meal, the matrix may act to absorb substances from the meal (e.g., carbohydrates) and release those substances to the body over a longer time period. Additionally, the fiber matrix may act to push ingested foodstuffs through the digestive tract faster.

In some embodiments, the effects of the nutrient fiber matrix (e.g., controlling blood sugar levels, insulin levels, fat levels, and/or body weight) are enhanced by the subject's diet. By way of example, the matrix may better control blood sugar levels when taken before the highest carbohydrate meal(s) of the day. In another example, the matrix may better control blood sugar levels when the subject reduces the consumption of sugary foods, processed foods, sweetened drinks, and/or snacks between meals.

In some embodiments, the effects of the nutrient fiber matrix are enhanced by the subject's activities. For instance, the effects of the matrix may be increased by raising the subject's physical activity level. In other instances, the amount of time that the subject waits between meals may also increase the effectiveness of the matrix.

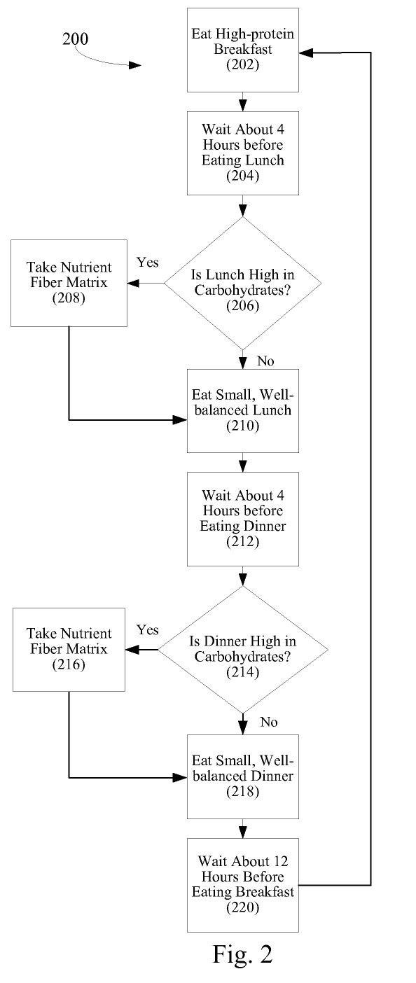

To better explain the methods for enhancing the effects of the fiber matrix by spacing the subject's meals, a typical example of an eating schedule, which has been found to be beneficial, is provided herein. Specifically, FIG. 2 shows a non-limiting example of a method 200 for scheduling meals and ingesting the matrix. Box 202 shows the subject begins the method by eating a high-protein, low-carbohydrate breakfast (e.g., oatmeal, eggs, bacon, protein drink, etc.). Then at box 204, the subject waits for at least approximately 4 hours before eating a snack (i.e., drinking a drink containing carbohydrates) or the next meal (e.g., lunch). As used herein, the term “approximately 4 hours” may refer to the preferred period of about 4 hours of time without food, which can be modified for particular needs and desires of the subject. By going approximately 4 hours without eating, the subject's body is given time to break down carbohydrates from the last meal and to decrease the subject's blood sugar levels. As the body's blood sugar levels decrease, the body's need to produce insulin may also decrease. Accordingly, the subject's body is given a period of time to metabolize the body fat.

When it is time for the next meal (e.g., lunch), box 206 shows that the subject may determine if the meal is to be high in carbohydrates. If the meal is high in carbohydrates or if the subject desires, the subject may ingest the nutrient fiber matrix before eating the meal, as shown at 208.

However, whether or not the subject takes the fiber nutrient matrix, box 210 shows that the subject continues to follow the method by eating a small, well-balanced lunch. As used herein the term “well-balanced” may refer to a nutritious meal that is neither high in proteins nor high in carbohydrates. For example, a well balanced meal may comprise one or more lean proteins, healthy fats, complex carbohydrates, fruits, and/or vegetables.

After the meal, box 212 shows that the subject continues the method by waiting for at least approximately 4 hours before eating a snack or the next meal (e.g., dinner). In some instances, the fiber nutrient matrix makes it easy for the subject to go without eating between meals. Indeed, as previously mentioned because the matrix can provide the subject with additional energy and cause the subject to feel satiated, the subject can comfortably go for longer periods without food than the subject could have gone without the matrix.

Box 214 shows that when it is time for the next meal (e.g., dinner), the subject determines whether the meal is to be high in carbohydrates. If the meal is high in carbohydrates, or if the subject desires, box 216 shows the subject continues the method by ingesting the nutrient fiber matrix before (e.g., about 5-15 minutes before) eating the meal.

Whether or not the subject ingests the matrix, box 218 shows the subject follows the method by eating a small, well-balanced meal (e.g., dinner). Following the meal, box 212 shows the subject may abstain from ingesting food for at least approximately 12 hours; after which time, the subject may eat another meal (e.g., breakfast). As used herein the term “approximately 12 hours” may be used to refer to the preferred period of about 12 hours of time without food, which can be modified for particular needs and desires of the subject.

Of course, the method 200 in FIG. 2 may be modified for specific schedules, needs, desires, and characteristics of an individual subject. For instance, while FIG. 2 illustrates that the subject may choose to ingest the nutrient fiber matrix before lunch and/or before dinner, the subject may also choose to ingest the matrix before breakfast or at any other suitable time. In some presently preferred embodiments, the subject ingests the fiber matrix two or more different times during a single day.

In other instances, the subject may modify the schedule set forth in FIG. 2 so that the subject can eat meals closer together. In one non-limiting example, the subject can wait at least approximately 3 hours after eating the first meal of the day before eating a second meal. In this example, the subject may also wait at least approximately 3 hours after eating the second meal before eating the third meal of the day. Moreover, in this example, the subject may wait at least approximately 10 hours after eating the third meal of the day before eating another meal.

In another non-limiting example, the subject can wait at least approximately 3.5 hours after eating the first meal of the day before eating a second meal. In this example, the subject may also wait at least approximately 3.5 hours after eating the second meal before eating the third meal of the day. Moreover, in this example, the subject may wait at least approximately 11 hours after eating the third meal of the day before eating another meal.

Additionally, while the method 200 in FIG. 2 illustrates that the subject goes without eating for about 4 hours between breakfast and lunch, about 4 hours between lunch and dinner, and about 12 hours between dinner and the next breakfast, in some instances, the subject may have a snack or drink, besides water, between meals. However, in such instances, it is preferred that the snack or drink be low in carbohydrates (e.g., a protein bar or shake) so as to not increase blood sugar levels and, hence, insulin levels, which may prevent the body from metabolizing body fat.

In some instances, the subject may further vary the schedule set forth in FIG. 2 by skipping one or more meals during the day. In such instances, the subject may take the matrix before one of the meals that the subject does eat, and/or the subject may ingest the matrix at a time when the subject does not plan on eating for an extended period of time.

In addition to the aforementioned benefits of the nutrient fiber matrix, the matrix may also offer several additional benefits. For example, consumption of the matrix may reduce the subject's sugar cravings. In another example, consumption of the matrix may prevent blood sugar levels from being volatile. Accordingly, the matrix may reduce the lack of energy and jitteriness associated with high or low blood sugar levels. In yet another example, the matrix can increase the subject's desire for healthy foods. In still another example, the matrix may increase the subject's body's ability to absorb beneficial nutrients. In still another example, the matrix may act to control lipid levels in the subject's body. In a final non-limiting example, the matrix may help increase the subject's immune system.

While specific embodiments of the present invention have been illustrated and described, numerous modifications come to mind without significantly departing from the spirit of the invention, and the scope of protection is only limited by the scope of the accompanying claims.

1. A method for controlling blood sugar levels in a mammal, the method comprising:
eating 3 meals during a first day with at least approximately 3 hours between the first meal of a first day and a second meal of the first day, at least approximately 3 hours between the second meal and a third meal of the first day, and at least approximately 10 hours between the third meal of the first day and the first meal of a second day; and
controlling blood sugar levels in the mammal by administering from about 3 g to about 56 g of a nutrient fiber matrix to the mammal during the first day.

2. The method of claim 1, wherein the nutrient fiber matrix comprises fiber, a plant extract, and a vitamin.

3. The method of claim 1, wherein the nutrient fiber matrix comprises guar gum, gum Arabic, locust bean gum, citrus pectin, oat fiber, maltodextrin, beta glucan, common bean extract, Aloe vera, broccoli powder, hydroxypropyl methylcellulose, biotin, vitamin B1, vitamin B2, vitamin B6, vitamin B12, and chromium.

4. The method of claim 1, wherein the first meal comprises a high-protein, low-carbohydrate meal.

5. The method of claim 1, wherein at least one meal comprises a high-carbohydrate meal.

6. The method of claim 5, wherein the nutrient fiber matrix is taken within about 30 minutes before, or during, the high-carbohydrate meal.

7. The method of claim 2, wherein the fiber is selected from a plurality of guar gum, gum Arabic, locust bean gum, citrus pectin, oat fiber, maltodextrin, and beta glucan.

8. The method of claim 2, wherein the plant extract is selected from a plurality of phaseolamine, broccoli powder, Aloe vera, hydroxypropyl methylcellulose, and ginseng.

9. The method of claim 2, wherein the vitamin is selected from a plurality of vitamin B1, vitamin B2, vitamin B6, and vitamin B12.

10. The method of claim 1, comprising administering from about 6 g and about 40 g of the nutrient fiber matrix during the first day.

11. The method of claim 1, comprising administering from about 14 g and about 30 g of the nutrient fiber matrix within about 30 minutes before, or during, at least one meal of the first day.

12. A method for controlling blood sugar levels in a person, the method comprising:
eating 3 meals during a first day with at least approximately 3.5 hours between the first meal of a first day and a second meal of the first day, at least approximately 3.5 hours between the second meal and a third meal of the first day, and at least approximately 11 hours between the third meal of the first day and the first meal of a second day; and
administering from about 6 g to about 40 g of a nutrient fiber matrix to the person during the first day, wherein the nutrient fiber matrix comprises fiber, a plant extract, and a vitamin, and wherein the first meal comprises a high-protein, low-carbohydrate meal.

13. The method of claim 12, wherein the fiber is selected from a plurality of guar gum, gum Arabic, locust bean gum, citrus pectin, oat fiber, maltodextrin, and beta glucan.

14. The method of claim 12, wherein the plant extract is selected from a plurality of phaseolamine, broccoli powder, Aloe vera, hydroxypropyl methylcellulose, and ginseng.

15. The method of claim 12, wherein the vitamin is selected from a plurality of vitamin B1, vitamin B2, vitamin B6, and vitamin B12.

16. The method of claim 12, wherein the nutrient fiber matrix further comprises chromium.

17. The method of claim 12, wherein the nutrient fiber matrix comprises guar gum, gum Arabic, locust bean gum, citrus pectin, oat fiber, maltodextrin, beta glucan, common bean extract, Aloe vera, broccoli powder, hydroxypropyl methylcellulose, biotin, vitamin B1, vitamin B2, vitamin B6, vitamin B12, and chromium.

18. A method for controlling blood sugar levels in a person, the method comprising:
eating 3 meals in a first day with at least approximately 4 hours between a first meal of the first day and a second meal of the first day, at least approximately 4 hours between the second meal and a third meal of the first day, and at least approximately 12 hours between the third meal of the first day and the first meal of a second day; and
administering from about 6 g to about 40 g of a nutrient fiber matrix to the person during the first day, wherein the nutrient fiber matrix comprises guar gum, gum Arabic, locust bean gum, citrus pectin, oat fiber, maltodextrin, beta glucan, common bean extract, Aloe vera, broccoli powder, hydroxypropyl methylcellulose, biotin, vitamin B1, vitamin B2, vitamin B6, vitamin B12, and chromium, wherein the first meal of the first day comprises a high-protein, low-carbohydrate meal, wherein at least one meal of the first day comprises a high-carbohydrate meal, and wherein the administration of the nutrient fiber matrix acts to control blood sugar levels in the person.

19. The method of claim 18, wherein the nutrient fiber matrix is taken within about 30 minutes before the high-carbohydrate meal.

20. The method of claim 18, comprising administering the nutrient fiber matrix to the person at 2 or more different times during the first day.
